(19) United States
(12) Patent Application Publication
(10) Pub. No.: US 2007/0082066 A1
(43) Pub. Date: Apr. 12, 2007

(54) Use of zeaxanthin to reduce light hyper-sensitivity, photophobia, and medical conditions relating to light hyper-sensitivity

(76) Inventors: Dennis L. Gierhart, Chesterfield, MO (US); Malcolm S. Grover, Chesterfield, MO (US)

Correspondence Address: JENKENS & GILCHRIST, P.C., 225 WEST WASHINGTON, SUITE 2600, CHICAGO, IL, 60606, US

(21) Appl. No.: 10/841,874

(22) Filed: May 7, 2004

Related U.S. Application Data
(60) Provisional application No. 60/468,517, filed on May 7, 2003.

(51) Int. Cl. A61K 36/82; A61K 36/45; A61K 33/32; A61K 31/7048; A61K 31/704

(52) U.S. Cl. 424/641; 514/690; 424/729; 424/752; 424/732; 514/27; 514/456; 514/763; 514/474; 514/458

(57) ABSTRACT

Zeaxanthin, a dietary carotenoid that occurs in very small quantities in corn, spinach, and other dietary sources, can reduce light hyper-sensitivity and various related conditions (such as migraine or other recurrent headaches) in people who suffer from it. Preferred dosages range from about 10 to about 100 mg per day, depending on the severity of condition, and the weight and medical status of the person. High dosages during an initial “buildup” dosing regimen can promote increased concentrations in the macula and lens, and lower dosages can then be used for maintenance purposes. Lutein may be able to achieve similar results, but because of certain molecular and cellular factors, zeaxanthin is preferable. Additional active agents (such as bilberry or other plant extracts, Vitamins C or E, zinc, etc.) can also be included or coadministered with any such formulations.

### RELATED APPLICATION

This application claims the benefit of a U.S. provisional patent application No. 60/468,517, filed May 7, 2003.

### BACKGROUND OF THE INVENTION

This invention is in the field of medicine and pharmacology, and relates to a dietary supplement that can be used to reduce and control a cluster of medical and physiological conditions referred to herein as “light hyper-sensitivity”. It is also referred to in numerous published articles as “chronic light hyper-sensitivity” (abbreviated as CLH).

This condition is also sometimes referred to as photosensitivity, but since everyone on earth is sensitive to light, in many ways, that term does not adequately convey the seriousness of the problems suffered by people who are hyper-sensitive.

The term “photophobia” is also sometimes used to refer to light hyper-sensitivity, but that term suffers from two drawbacks. For one thing, the term “phobia” tends to suggest, in the minds of most people, a form of irrationality that usually involves a severe neurosis or even a mental illness, and light hyper-sensitivity is not a mental disorder. In addition, scientists and physicians do not use the term “photophobia” consistently. Some scientists and physicians use “photophobia” as equivalent and interchangeable with light hyper-sensitivity; however, other scientists and physicians use “photophobia” to refer only to cases that are sufficiently severe to drive a person to seek help, either through medical attention, or by adopting various personal routines, such as always putting on sunglasses before stepping outside. Still other scientists and physicians use “photophobia” to refer only to cases of light hyper-sensitivity that are so severe that they require medical intervention (as examples, some people suffer from migraine headaches, epileptic seizures, or other disorders that are triggered by various types of lighting conditions).

As a rule, any condition that can be fairly diagnosed and labeled as a form of “hyper-sensitivity” will involve a large and unavoidable subjective component, since the hypersensitive condition will involve variations in how a particular individual perceives, processes, and responds to inputs that trigger sensory or nociceptive (pain-signalling) neuronal networks. Just as with a neuropathic pain condition, the only person who can really know and understand the severity of a particular case is the person who suffers from it. Because of the wide range of variability in how people perceive and respond to things, there are no adequate ways to quantify or define a boundary line where a “normal” person would find a certain problem or irritant to be tolerable, while a “hyper-sensitive” person would be driven to serious or intense discomfort by the same problem or irritant.

An illustration of the range and variability of light hyper-sensitivity can be provided by the following example. Throughout America and the rest of the world, millions of people drive in a generally eastward direction, to get to work each morning, and they drive in a generally westward direction, to get back home each evening. During both drives, during at least some months as the seasons change, the sun will be relatively low in the sky, and those drivers must look generally in the direction of the sun, while they drive to and from work. Even if they use the car visors and sunglasses, they still must deal with glare, and with occasional periods of sunlight shining in their faces and eyes.

Most people who make that kind of drive, five days a week, learn to do so without suffering from serious problems or annoyance. But for thousands of people, driving toward the sun when it is low on the horizon is unusually difficult and unpleasant, and highly stressful. It saps their energy and enthusiasm, and it often drives and pushes them toward a headache, making their workdays substantially less pleasant and productive, after they arrive at the office. It also tends to make them more tired, irritable, and unpleasant, after they arrive back home in the evening.

These people can fairly be said to suffer from light hyper-sensitivity. It is entirely subjective, and not everyone suffers from it. However, it is entirely real and serious, to anyone who suffers from it. Many thousands and perhaps even millions of people around the world, who suffer from light hyper-sensitivity and who cannot avoid situations where it arises without suffering a substantial loss of income, autonomy, interactions with other people, or other desirable things, would do anything within reason, if they could find away to make their light hyper-sensitivity condition less severe, less stressful, and less unpleasant.

In addition, in many people, light hyper-sensitivity reaches levels that seriously impede or destroy their ability to function at a desired level, as a pleasant and productive coworker or family member, or their ability to enjoy activities such as golf, fishing, skiing, watching a sports event, etc. Light hyper-sensitivity can also interfere with performance in various sports and other outdoor activities. Similarly, in many people who suffer from recurrent migraine headaches, sunlight or other bright lights (and in some cases, blinking or moving points of lights, as occur during nighttime traffic) can become “triggering factors” that can provoke the onset of an excruciating and debilitating headache that will force the person to stop whatever he or she is doing and go lie down.

In addition, many people with epilepsy can have seizures triggered by bright or flickering lights. Photosensitive epilepsy is the most common of the “reflex” epilepsies, and has been estimated to be a triggering factor in 10% of all seizures in young people with epilepsy (e.g., Harding et al, “Two visual mechanisms of photosensitivity,” Epilepsia 40: 1446-51 (1999)). In many people with epilepsy, especially among children, seizures can be triggered simply by watching a television.

Accordingly, light hyper-sensitivity is a serious problem among many people, and currently, there are no fully adequate preventive treatments. Various medicines can help control certain cases or symptoms (for example, among epileptics, anti-seizure drugs usually can be fairly effective in preventing most light-induced seizures); however, those types of medicines are not suited for the majority of patients who fall into non-acute categories (such as, for example, someone who tends to get a headache and/or suffer from fatigue, after looking toward the sun while driving to or from work).

In addition, among epileptics and others who are forced to take an anti-seizure or other neuroactive drug, on a chronic basis, in order to reduce the frequency or severity of light triggered seizures or similar adverse events, such neuroactive drug often have their own drawbacks and unwanted side effects.

In addition, a number of potentially useful implications would arise, if people who must work or otherwise perform in occasionally dark conditions could be provided with reduced glare sensitivity, and/or improved ability to recover visual acuity rapidly, after flashes of bright lights at night. Airline or military pilots, soldiers, cargo handlers, and various other occupations and endeavors offer examples of such potential benefits.

Accordingly, it would be an important and useful advance if a healthy and nutritious dietary supplement that does not cause any known drawbacks or unwanted side effects, and which instead helps promote better overall health (and better eye health and skin protection, in particular), were determined to effectively reduce light hyper-sensitivity, among people who suffer from that condition.

Based on all evidence gathered to date, it appears that such a discovery has indeed been made, involving a naturally-occurring and nutritious carotenoid called zeaxanthin. This carotenoid, and a closely similar carotenoid called lutein, is described in the following section.

Zeaxanthin and Lutein

Zeaxanthin and lutein are two carotenoids that occur naturally in various types of plants and bacteria. Their classification as carotenoids generally implies three chemical and physical properties: (1) they are usually synthesized in nature by coupling together units of a precursor called isoprene (also called isopentenyl), an unsaturated molecule with 5 carbon atoms; (2) they absorb high-energy wavelengths, in the blue and ultraviolet region of the spectrum, and therefore, carotenoids usually appear as red, orange, or yellow pigments, since those longer wavelengths are reflected rather than absorbed; and, (3) they are anti-oxidants, which means they can absorb and “quench” various types of destructive oxidative free radicals, to prevent the oxidative radicals from attacking and damaging cell membranes, proteins, DNA, and other biomolecules.

Since carotenoids are effective UV absorbers and anti-oxidants, they evolved over billions of years as compounds used by plant and bacteria that must be able to survive in conditions of prolonged exposure to direct sunlight (which carries levels of ultraviolet radiation that otherwise will kill cells, mainly by randomly breaking apart molecules such as proteins and DNA). Since carotenoids cannot be synthesized by animals, animals must obtain adequate supplies of carotenoids in their diets, in a manner comparable to vitamins. Indeed, certain carotenoids (such as beta-carotene, a precursor molecule that is broken in half to release Vitamin A) are indeed formally recognized as vitamins.

The structures of zeaxanthin and lutein are shown in FIG. 1, along with beta-carotene, which does not contain any hydroxy groups on its end rings. Zeaxanthin and lutein are isomers of each other, differing only in the placement of a double bond in one of the end rings. Their biochemical activities are described in more detail in various published items, including U.S. reissue Pat. Re-38,009 (Garnett et al 2003) and other articles cited therein.

Over the past two decades, zeaxanthin and lutein have become of interest to people studying vision and eyesight, since they are the two carotenoid pigments that impart a yellow color to a small round spot called the macula, located at the center of the retina, in humans and other primates (however, dogs, cats, rodents, and most other animals do not have a macula). The macula is crucial for clear vision in humans, and studies have indicated that people who do not obtain enough zeaxanthin and lutein in their diets tend to suffer higher risks of an eye disease called macular degeneration.

Therefore, lutein and zeaxanthin pills have been developed, for treating or preventing macular degeneration in humans.

Bulk lutein has been available for roughly 20 years, since it is present in reasonably high concentrations in the orange petals of marigold flowers. It was initially developed and commercialized as a pigmenting additive for chicken and salmon feeds, since it can cause the skins of chickens and the meat of farm-raised salmon to turn a darker, richer, more appealing color (although it often must be supplemented by a red pigment as well, to achieve that desired effect). In the U.S., lutein is sold in bulk by Kemin Foods (Des Moines, Iowa; www.keminfoods.com), and by Cognis (www.cognis.com). Because of its recently recognized potential in treating or preventing macular degeneration in humans, it is now also being sold for human use, both in low dosages as one component in multivitamin formulations (such as Centrum, sold in the US by Wyeth Consumer Healthcare, a division of American Home Products), and in higher-dosage stand-alone products sold by various smaller companies, such as DreamPharm, Now Foods, and VitaminLab (any of these companies and their products can be located easily by an Internet search for “lutein”).

Zeaxanthin is much rarer in nature, and there is no convenient bulk source, comparable to lutein from marigolds. Therefore, zeaxanthin supplements were not available in any quantities higher than “trace” amounts, until early 2002; prior to that, the only sources were “mixed carotenoid” preparations, which in most cases were little more than dried flakes of spinach, kale, and other dark green vegetables, occasionally supplemented by dried flakes from carrots, red peppers, etc. In late 2001, Roche Vitamins, Inc. (which recently has been acquired by DSM Chemicals, headquartered in Holland) began selling bulk quantities of synthetic R,R-zeaxanthin to formulators and retailers. The main retail seller is ZeaVision LLC (St. Louis, Mo.; www.zeavision.com), which is the assignee and owner of above-cited U.S. Pat. Re-38,009 and certain other patents as well, in the U.S. and elsewhere. Zeaxanthin can also be obtained from certain types of marine algae, from a Flavobacterium multivorum bacterial strain identified and patented by Gierhart, and from certain types of berries, often called “Goji” berries.

It should also be noted that zeaxanthin and lutein are also present in the lenses of human eyes, and various reports suggest that they appear to have two benefits. First, they help absorb ultraviolet radiation, to prevent it from reaching and damaging the retina. Second, they may also help prevent or reduce cataract formation. Those roles and activities in the lens are more difficult to optically measure and quantify than pigment density in the macula, and unlike macular degeneration, lens damage can be repaired through lens replacement, in cataract surgery. Therefore, the roles, activities, and benefits of zeaxanthin and lutein in the lens have not been studied as extensively as their presence and roles in the macula.

Despite their utility in treating and preventing macular degeneration, neither zeaxanthin nor lutein has previously been recognized as having an additional potential utility, in treating the class of problems referred to herein as light hyper-sensitivity, described above.

Accordingly, one object of this invention is to disclose that zeaxanthin (and possibly lutein, although probably to a lesser extent and with less potency) can be effective, in at least some people, in reducing light hyper-sensitivity. As such, it can be used by people whose daily routines (such as driving toward the sun while it is low in the sky), pleasure outings (such as fishing, golf, skiing, vacations, etc.) or other activities, are impeded and rendered less enjoyable because of sunlight conditions, or who must travel or work under sunlight or other lighting conditions that cause or aggravate stress or headaches. It can be used to treat severe cases of light hyper-sensitivity, including severe cases that can be fairly diagnosed as photophobia.

Another object of this invention is to disclose that zeaxanthin, and possibly lutein as well, can be effective, in at least some patients, in treating at least some cases of light hyper-sensitivity that arise from, or are related to, various ocular problems, such as cataracts, glaucoma, diabetic or other retinopathy, and corneal problems, including injuries, abrasions, infections, and problems related to contact lenses, or refractive surgery to correct near or far-sightedness.

Another object of this invention is to disclose that zeaxanthin, and possibly lutein as well, can be effective, in at least some patients, in treating at least some cases of light hyper-sensitivity that arise from, or are related to, various other medical conditions, such as albinism, conditions related to drugs that can cause or aggravate light hyper-sensitivity, and cases of light hyper-sensitivity that appear to be related to aging or other medical conditions, including conditions that require chemotherapy.

Another object of this invention is to disclose that zeaxanthin, and possibly lutein as well, appears to be effective, in at least some patients, in reducing the frequency, severity, and/or duration of some types of recurrent headaches, including migraine headaches triggered by direct or intense sunlight, and possibly including some cluster headaches and/or tension headaches in at least some patients.

Another object of this invention is to disclose that zeaxanthin, and possibly lutein as well, is likely to be effective, in at least some patients, in reducing the frequency, severity, and/or duration of epileptic seizures that are triggered or aggravated by certain types of bright or flickering lights.

These and other objects of the invention will become more apparent through the following summary and description of the preferred embodiments.

### SUMMARY OF THE INVENTION

Zeaxanthin has been discovered to be effective, in at least some people, in reducing light hyper-sensitivity. Preferred dosages for such use are in the range of about 10 to about 100 mg per day, depending on the severity of the hypersensitive condition, and the size and weight, condition, and medical status of the person taking it. High dosages can be used to treat severe cases, including cases that can be fairly diagnosed as photophobia, and dosages as low as 1 mg/day may provide significant help to at least some people.

People who can also benefit from this treatment include: (1) people who suffer from light hyper-sensitivity due to ocular problems, such as cataracts, glaucoma, diabetic or other retinopathy, and corneal problems, such as injuries, abrasions, infections, and problems related to contact lenses or laser surgery; (2) people whose daily routines, pleasure outings or vacations, or other activities are impeded or rendered less enjoyable because of sunlight conditions; (3) people who must work or carry out other activities under lighting conditions that create or aggravate stress, headaches, or other problems; (4) people who suffer from migraine or other recurrent headaches, epileptic seizures, or other episodic problems that can be triggered or aggravated by certain types of lights; and, (5) people who suffer from albinism, conditions related to drugs that can cause or aggravate light hyper-sensitivity, and cases of light hyper-sensitivity that are related to aging or other medical conditions.

Because of its chemical and structural traits, and because it is naturally deposited in high quantities in the very center of the human macula, zeaxanthin is believed to be ideal for treating and reducing light hyper-sensitivity. Although lutein (a structurally similar isomer of zeaxanthin) is believed to have less efficacy than zeaxanthin at reducing and controlling light hyper-sensitivity and photophobia, it nevertheless may be able to offer some relief in some patients, and it is already present in most natural food or pigment sources that contain zeaxanthin. Accordingly, lutein (especially lutein preparations that naturally contain some level of zeaxanthin, such as marigold flower extracts) can be evaluated in any particular patient, if desired, to determine whether it can help reduce light hyper-sensitivity in that patient.

### BRIEF DESCRIPTION OF THE DRAWINGS

FIG. 1 shows the chemical structures of beta-carotene, zeaxanthin, and lutein, indicating the “misplacement” of the double bond in one end ring of lutein, which creates a non-symmetric molecule with a disrupted “conjugated electron cloud” over that end ring.

### DETAILED DESCRIPTION

Zeaxanthin has been discovered to be effective, in at least some people, in reducing light hyper-sensitivity. As used herein, the term “light hyper-sensitivity” includes conditions that are triggered or aggravated by direct sunlight or other bright, flickering, or other light exposures or patterns, which induces visual stress of a type that generates a serious and substantial degree of fatigue, annoyance, or other problems (including headaches, seizures, etc.) that are sufficiently severe to cause the sufferer to seek medical relief or other active intervention, above and beyond the wearing of sunglasses or other protective lenses. Light hyper-sensitivity also includes problems relating to glare, and impaired glare recovery.

While some people may benefit from dosages as low as 1 mg/day, preferred dosages for treating people who suffer from this condition are in the range of about 3 to about 100 mg per day. These dosages can be conveniently achieved via 10 mg capsules, which are available from ZeaVision LLC, the assignee herein (www.zeavision.com). Because carotenoids are fat-soluble hydrophobic compounds that do not dissolve in water, any oral ingestion of such capsules preferably should accompany a meal, so that the normal secretion of bile by the digestive tract can help increase the absorption and uptake of zeaxanthin into circulating blood. Although the current versions of such capsules contain gelatin-coated beadlets, steps are being taken to develop softgels or similar capsules that will contain an oily carrier substance, such as olive oil. Similarly, a preferred mode of ingestion is in a foodstuff, such as (i) a fortified food, such as margarine, a dairy product, a beverage, a meat product that does not require harsh cooking, etc., or (ii) a food additive, such as a powder, syrup, or other formulation that is designed to be added to a beverage, soup, salad, or other food substance.

Because light hyper-sensitivity has a large subjective component, and becomes a substantial problem if and to the extent that a person finds it so irritating and annoying that it requires active steps to either avoid it or treat it, the method of this invention, and various claims set forth below, must also take into account that subjective element. This is especially true, since this invention can be carried out using “over-the-counter” nutritional supplements containing zeaxanthin, which is naturally present in the diet and available to the public in supplement form, without a prescription, under the provisions of the Dietary Supplement Health and Education Act.

Accordingly, the method of this invention can be carried out either with or without formal medical diagnosis or supervision, such as by someone who knows that he or she has a problem with unusual sensitivity to light (accompanies by headaches, fatigue, etc.), and who hears about this type of zeaxanthin treatment from an ophthalmologist, optometrist, or physician, or from a friend, relative, news article, website, or other source.

Accordingly, in one preferred embodiment, this invention discloses a method for reducing light hyper-sensitivity, comprising the step of administering, to a person in need of such treatment, zeaxanthin at a dosage that reduces such light hyper-sensitivity, to an extent that is noticeable and apparent to such person. The phrase “noticeable and apparent” requires that such relief and/or other benefits (such as reductions in the number of headaches, etc.) rise to a level that is clearly recognizable to and appreciated by the person with the light hyper-sensitivity problem.

Such dosage normally will be achieved by daily or other periodic or intermittentoral ingestion, preferably at a relatively high “buildup” dosage for about 2 to 4 weeks, followed by a lower long-term “maintenance” dosage.

In most people who suffer from mild to moderate light hyper-sensitivity, dosages in the range of about 10 to 40 mg/day are likely to be preferred, especially during an initial dosing period, while levels in the blood serum are increasing, and leading to deposition of additional quantities of zeaxanthin in the macular portion of the retina. After initial dosing levels have generated higher concentrations of zeaxanthin in the macula, somewhat lower maintenance dosages (such as about 6, 3, or possibly even 1 mg/day) are likely to be sufficient.

Since 20 mg/day dosages have been shown to be safe and well-tolerated by thousands of users who have been taking zeaxanthin for macular degeneration over the past few years, a 20 mg/day dosage level is regarded as providing a suitable and preferred trial dosage, for any person who is initially trying such a treatment to determine whether it will help resolve or reduce a light hyper-sensitivity condition. If no results are noticed at that level within about 2 to 4 weeks, the person can try a somewhat higher dosage during the next 2-4 weeks.

Dosages of 50 or more mg/day can be used to treat severe cases, including cases that involve serious medical concerns, such as light-triggered migraine headaches or epileptic seizures, and cases involving albinism, or ocular problems such as cataracts, glaucoma, diabetic or other retinopathy, and corneal problems, including injuries, abrasions, infections, and problems related to contact lenses or laser surgery.

If desired, any dosage regimen can be accompanied by diagnostic procedures, such as non-invasive retinal photographs or analyses that can be used to analyze “macular pigment optical density” (MPOD). Several techniques for measuring MPOD are known, including flicker photometry, spectral fundus reflectometry, and scanning laser ophthalmoscopy (e.g., using methods such as described in Berendschot et al 1999, Investig. Ophtihalm. Vis. Sci. 40: S314, 1999). Accordingly, a preferred dosage for any particular patient is a daily dosage that will initially create, and then sustain over a long period of time, a significant increase in MPOD, for that particular patient; as described above, such dosages may comprise a relatively high “buildup” dosage, followed by a lower “maintenance” dosage.

If desired, such treatment can be accompanied by various types of medical supervision, diagnostic tests, MPOD measurements, etc., or by a patient keeping daily logs or other records of the extent to which light-sensitivity problems are perceived by the person receiving the treatment.

Alternately, rather than regarding this type of method as being carried out by “administering” zeaxanthin to a person (that term tends to suggest and imply an intervention, supervision, or other action by an additional person), the invention can be regarded as a method for reducing light hyper-sensitivity, in a person in need of such treatment, comprising the step of orally ingesting zeaxanthin, at a dosage that reduces such light hyper-sensitivity to an extent that is substantial and readily noticeable to such person.

This treatment is also likely to be able to provide at least some benefit to at least some people in any or all of the following categories: (1) people who tend to suffer from headaches, serious fatigue, or other comparable problems, either while they drive or shortly after driving, especially if they must drive in a direction that subjects them to high levels of glare, sunlight directly in their face, or similar conditions; (2) people who work or conduct other activities under lighting conditions that cause or aggravate stress, headaches, fatigue, or other problems; (3) people whose daily routines, pleasure outings, vacations, or other activities are impeded or rendered substantially less pleasant and enjoyable because of eye or vision strain relating to direct sunlight or other lighting conditions; and, (4) people who are preparing for a vacation, trip, or other outing or activity that will involve sustained sun exposure or unusual lighting (such as, for example, a fishing, hunting, camping, or golf outing, a skiing or boating outing, a trip to a location such as an amusement park, water park, etc.).

The Applicants herein did not set out to study and evaluate light hyper-sensitivity, as a particular condition that might be treated by zeaxanthin. Instead, this discovery arose out of observations that were made while zeaxanthin was being tried and evaluated by a number of people, to determine whether it could help protect the skin against sunburns. Because of various factors and events described in a separate patent application (Ser. No. 10/356,134, Gierhart et al; the contents of that application are incorporated herein by reference, as though fully set forth herein), the founder of ZeaVision (who is also one of the Inventors herein) became convinced that relatively high dosages of zeaxanthin, in the range of about 30 mg or greater per day, could increase the skin's ability to withstand direct sunlight without suffering a sunburn. Accordingly, he and several coworkers began taking zeaxanthin at dosages in a range of about 30 to about 80 mg/day, during the summer of 2002, to provide additional evaluation of the earliest results that had been seen.

The results of those tests, using summertime exposure to direct sunlight, confirmed that high dosages of zeaxanthin can indeed reduce the risk of sunburns. Therefore, a patent application entitled “Protection Against Sunburn and Skin Problems with Orally-Ingested High-Dosage Zeaxanthin”, was drafted and filed in the autumn of 2002, as a provisional application, based on anecdotal evidence from several people. Subsequently, indoor tests during the winter season, using a medical-grade ultraviolet lamp to measure before-and-after “minimal erythemal dosages” (a standardized measurement used by dermatologists and researchers), provided objective and verifiable data, which strongly confirmed the skin-protective effects of high-dosage zeaxanthin. After those test data became available, the provisional patent application on sunburn prevention was expanded into a utility application.

As an unexpected byproduct of the use of high-dosage zeaxanthin by several people working with ZeaVision (and by certain other people, such as family members and friends who learned of its benefits in protecting against sunburns, after the patent application had been filed), it was noticed, as the days grew longer and the sunlight grew more intense during the spring of 2003, that several people who normally had worn sunglasses almost all the time whenever driving or outdoors, had stopped using their sunglasses so much. When asked about it by the Applicants herein, they offered answers that were usually along the lines of, “I hadn't noticed that. But yeah, you're right. I haven't been wearing my sunglasses as much as I used to. I just don't feel like I need them as much, or as often.”

At roughly the same time, the customer service specialist at ZeaVision also began to receive sporadic comments from people who had purchased zeaxanthin capsules to treat or prevent macular degeneration. Several such consumers volunteered comments to the effect that they seemed to be suffering less from light hyper-sensitivity, after taking zeaxanthin, than before. When asked for any more details, most such consumers indicated they had simply assumed that light hyper-sensitivity was part of having macular degeneration, and as their macular degeneration condition began to improve (or at least stabilize) once they began taking zeaxanthin, the accompanying light hyper-sensitivity condition also improved.

After those anecdotal responses were considered, searches of medical databases were carried out on photophobia and light hyper-sensitivity, to evaluate the nature, frequency, and severity of the problem. The published reports that were located and reviewed indicated that those are substantial problems, and they rise to a level of genuine importance among many people, especially people who suffer from migraine headaches, epileptic seizures, or other medical problems that can be triggered or aggravated by unwanted exposure to various types of intense, flickering, or other lights.

These discoveries led the customer support staff at ZeaVision to begin routinely asking customers about light hyper-sensitivity, if they called in and wanted to discuss the visual improvements they were enjoying after they began taking zeaxanthin. When that type of questioning by the customer service specialist became routine, and he began keeping notes of such conversations, it emerged that roughly 30% of the customers who were asked those types of questions indicated that they had indeed been suffering from unusual and unpleasant levels of light sensitivity, and nearly all of those patients indicated that it had grown less severe and less troubling, after they began taking zeaxanthin capsules. As with the earlier reports, most such consumers had simply assumed that light hyper-sensitivity was simply part of having macular degeneration, and as their macular degeneration condition began to improve once they began taking zeaxanthin, the accompanying light hyper-sensitivity condition also improved. However, when asked to consider and evaluate any unusual, irritating, or otherwise noticeable light sensitivity, separately and independently from vision clarity (as would be measured by an optician during an eye exam), nearly all patients who had reported an unwanted increase in light sensitivity readily and promptly agreed that it had improved, significantly, since they had started taking zeaxanthin at 10 mg or more per day.

In addition, a number of customers indicated that they suffered from headaches with a severity that would be regarded as migraines (or which had been diagnosed by a physician as migraine headaches), and/or with a frequency that would be regarded as recurrent. When asked about any recent changes in the frequency or severity of such headaches, a number of those customers reported that they didn't seem to be having as many migraine or other severe headaches, after commencing zeaxanthin at 10 mg or more per day.

Based on the results of those reports by purchasers and users of zeaxanthin, it is believed by the Applicants herein that zeaxanthin, in addition to its benefits in treating or preventing macular degeneration, and in reducing or preventing sunburns and certain other skin problems, can also be effective, in at least some patients, in reducing light hyper-sensitivity (including cases that trigger migraine or other recurrent headaches or other problems, and severe cases that can fairly be diagnosed as photophobia).

Based on those findings, ZeaVision has taken steps to organize and contribute to the funding of human clinical trials, using patients who have been diagnosed as suffering from any of several known types of light hyper-sensitivity, at two different optometry schools. Although it will take some period of months before the findings become known, the design of those trials should be noted. Briefly, these clinical trials (as currently being planned) will involve 30 patients each, who have complained to their optometrists or ophthalmologists about light hyper-sensitivity, but who have no other known ocular disease or disorder. Each patient will be tested for visual acuity, contrast sensitivity, glare recovery, and other indicators of visual health, and records will be kept of any related symptoms (such as frequency and severity of migraine headaches, etc.). “Macular pigment optical density” will also be measured. The patients will be given 20 mg of zeaxanthin each day, and monthly measurements will be made of the same parameters mentioned above, to see whether they improve over time.

Unless and until the results of clinical trials indicate otherwise, this type of benefit from zeaxanthin supplements, for people who suffer from light hyper-sensitivity, is believed and presumed to be able to benefit at least some people suffering from light hyper-sensitivity, regardless of the etiology, symptoms, manifestations, or accompanying medical problems involved in a particular case. For example, various ocular problems, including cataracts, glaucoma, and diabetic or other retinopathy, are known to cause or aggravate light hyper-sensitivity, and corneal problems (including injuries, abrasions, infections, and problems related to contact lenses or laser surgery) are also known to cause or aggravate light hyper-sensitivity. Accordingly, anyone who is suffering from any of those conditions can be treated with zeaxanthin, at a trial dosage regimen such as 20 to 30 mg/day for at least two weeks and preferably a month, to see whether the problem abates in that particular individual.

Similar treatments using zeaxanthin are also likely to be effective, in at least some patients, in reducing the frequency, severity, and/or duration of recurrent headaches (such as migraine headaches, and possibly cluster and/or tension headaches).

In addition, similar treatments using zeaxanthin are also likely to help ameliorate certain other medical conditions that involve light hyper-sensitivity as a causative, contributing, triggering, or aggravating factor; such conditions include, for example, epileptic seizures that are triggered, in some patients, by exposure to certain types of bright and/or flickering lights.

In addition, similar treatments using zeaxanthin may also be able to provide benefits to people suffering from various other classes of known ailments, if such ailments have one or mole symptoms suggesting that light hyper-sensitivity may be a causative, contributing, triggering, or aggravating factor. One example includes people suffering from chronic fatigue syndrome; in at least some of those patients, the fatigue they are feeling may be aggravated, to some extent, by light hyper-sensitivity. Similarly, people who suffer from a chronic or intermittent neurological condition or syndrome that tends to grow more severe and less controllable when the person is suffering from serious stress or fatigue (such as Tourette's syndrome, as one example), may also benefit from zeaxanthin supplements, if light hyper-sensitivity is contributing to the stress and fatigue levels that aggravate that condition or syndrome in that person.

Accordingly, oral ingestion of zeaxanthin supplements, in a daily dosage in a range of about 10 to about 100 mg/day, can be tested and evaluated by any patient suffering from an unwanted medical condition that is suspected of being caused or aggravated by, or associated with, light hyper-sensitivity.

It should also be noted that any such zeaxanthin supplement may also contain any other active agent that is known to (or believed to) help support eye health. Such additional agents include for example, lutein, extracts from bilberries, wolfberries, gingko biloba, green tea, or various other plants, anti-oxidants such as Vitamins C or E, zinc, and various compounds from soybeans and other plants, that are often referred to a flavonoids, flavones, isoflavones, polyphenols, anthocyanins, etc.

Evaluating Lutein for Controlling Light Hyper-Sensitivity

As mentioned above, zeaxanthin and lutein are the only two carotenoids that are Present in any significant quantities, in the yellow-colored macula portion of the human retina. Therefore, now that preliminary testing and consumer inquiries have indicated that zeaxanthin is effective in reducing and controlling light hyper-sensitivity, questions arise as to whether its isomer, lutein, can also help reduce and control light hyper-sensitivity.

An informed appraisal of that question and issue requires an understanding of: (1) the chemical differences between zeaxanthin and lutein; (2) the resulting differences in their performance traits; and, (3) the patterns of deposition of both zeaxanthin and lutein, in the macula.

As briefly mentioned above, zeaxanthin is much rarer than lutein in dietary sources; however, the human retina deposits higher concentrations of zeaxanthin than lutein in the center of the macula, which is crucial for clear vision. This unusual pattern of deposition, in human retinas, is believed to arise because of certain molecular factors that cause zeaxanthin to be more effective than lutein, in both: (i) absorbing potentially harmful UV light, and (ii) quenching oxidative free radicals.

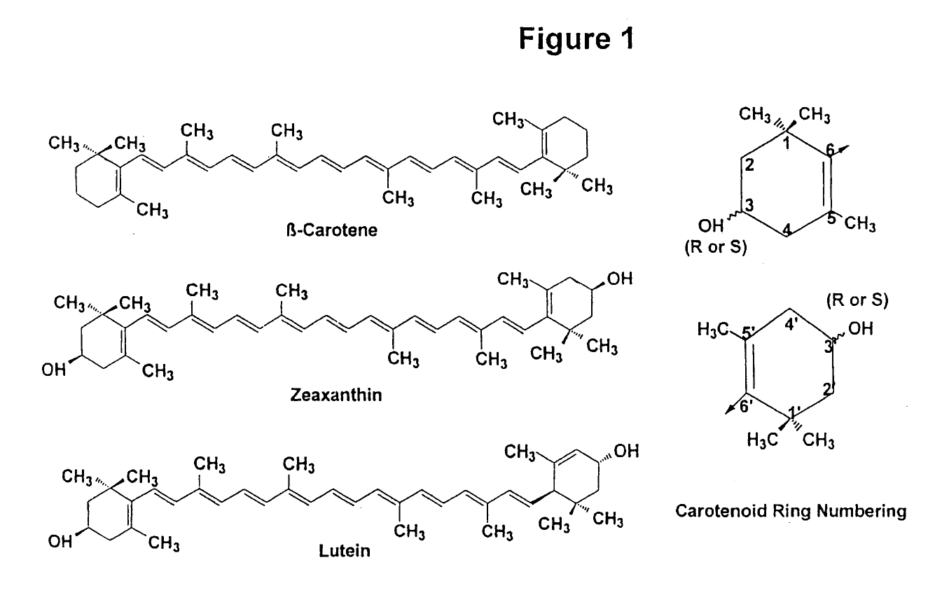

As can be seen by inspecting the end rings of the chemical structures drawn in FIG. 1, there are two important molecular differences between zeaxanthin and lutein:

(1) Zeaxanthin is completely symmetric. Both of its end rings have a ring structure called a “beta” ring, with a certain placement of the double bond in the ring. This same ring structure is also found at both ends of beta-carotene, which is also shown in FIG. 1. By contrast, lutein is not symmetric. It has a beta ring at one end, but it has an “epsilon” ring at the other end. The symmetry of zeaxanthin, and the lack of symmetry of lutein, may contribute to the deposition of higher quantities of zeaxanthin, than lutein, at the center of the macula.

(2) Because of the placement of the double bonds, a “beta” ring has a higher degree of conjugation than an epsilon ring. Therefore, zeaxanthin has a higher level of conjugation than lutein.

In this context, “conjugation” refers to the placement of alternating single and double bonds, along a chain of carbon atoms that are bonded together. If single and double bonds are placed in a series, alternating with each other, an unusual result arises. An electron “cloud” is formed, which is not pinned down to specific atomic locations, and which instead acts in a more fluid, movable, and adaptable manner, to cover the entire conjugated portion of a molecule. This type of “conjugation” also occurs in benzene rings, which have alternating single and double bonds in a six-carbon ring; instead of creating fixed electron locations, benzene becomes a flat planar molecule, with a ring-shaped electron cloud that hovers next to each side of the carbon ring.

The electron “cloud” that covers the conjugated portion of a carotenoid molecule is very effective at absorbing and neutralizing high-energy UV photons, without being damaged by those otherwise destructive photons. In a sense, this is analogous to a cushion or a pillow being able to absorb a hard impact from a boxing glove or baseball bat, even though that same impact would break a wooden board that cannot flex, compress, and yield.

Similarly, the conjugated cloud that surrounds most of a carotenoid molecule is also the part that is able to absorb, “quench”, and neutralize oxidative free radicals, to prevent them from attacking and damaging other molecules.

This leads to a crucial difference between zeaxanthin, and lutein. The conjugation of zeaxanthin extends all the way into both of its end rings. By contrast, lutein does not have any conjugation covering part of its epsilon ring.

That factor probably would be significant, under any condition. However, it is elevated and magnified to a level of crucial, critical, and vital importance, in animal cells, because of the way that lutein and zeaxanthin molecules position and align themselves in cell membranes, in a manner often referred to as “membrane-spanning”.

Because of chemical factors that are explained in nearly any textbook on cells or biology, the outer membrane of any animal cell is a “lipid bilayer”, made of molecules that have water-soluble amino-phospho “heads”, and water-insoluble lipid “tails”. Two layers of these amino-phospho-lipid molecules will line up together, in a way that places the oily, fatty, hydrophobic “tails” in the protected center of the membrane, so they will not have to be in contact with water. The water-soluble “heads” line up in a manner that creates the water-contacting surfaces, on the two opposing sides of the bilayer membrane. One layer of these hydrophilic “heads” creates the outer surface of a cell membrane, and interacts with the watery (“extracellular”) fluids that surround the cell. The other layer of hydrophilic “heads” faces inward, and interacts with the watery fluid (cytoplasm) contained inside the cell. The center layer of the membrane is an oily, fatty, hydrophobic layer that minimizes any contact with watery liquids.

In both zeaxanthin and lutein, the center chain (i.e., the zig-zagging portion that forms a bridge between the two end rings) is an oily, fatty, hydrophobic domain, which does not mix with water. Therefore, that center chain aligns itself so that it can remain inside the fatty layer that resides inside the middle of a cell membrane. Indeed, since beta-carotene has no hydroxy groups on either end ring, it aligns itself completely within the interior of a call membrane.

However, because of the hydroxy groups attached to their end rings, zeaxanthin and lutein tend to line up in a manner that causes them to “span” (or straddle) a cell membrane of a cell. The center chain lines up in a “radial” direction (comparable to the spokes on a wheel), and a portion of each end ring becomes exposed and accessible, on the inner and outer surfaces of the cell membrane (because of evolution and natural selection, it is not merely a coincidence that carotenoids created by adding oxygen molecules or hydroxy groups, to beta-carotene end rings, have molecular lengths that are perfectly suited to enable the end rings to protrude out from both sides of an animal cell membrane).

Since zeaxanthin has a conjugated (i.e., UV-absorbing, anti-oxidant, cell-protective) “electron cloud” that extends over and partially covers both of its end rings, while lutein has a conjugated (UV-protective, anti-oxidant) electron cloud over a portion of only one of its end rings, that apparently minor structural difference apparently was sufficient, over the course of evolution, to drive the retina to develop ways to deposit high concentrations of zeaxanthin at the crucial center portion of the macula, despite its rarity in food sources. By contrast, lutein (even though it is much more common and widespread in food sources) is deposited in higher quantities around the periphery of the macula.

Another factor is also worth noting, concerning the way the retina uses zeaxanthin and lutein. Using mechanisms that are not fully understood, and that presumably involve enzymatic and/or light-triggered chemistry, a substantial portion of the lutein in human retinas is converted into an isomeric form of zeaxanthin that does not exist in any known food supplies. That isomer, which can be called 3R, 3′S-zeaxanthin, is also called meso-zeaxanthin. This can be regarded as suggestive evidence that the retina appears to prefer zeaxanthin over lutein.

Finally, it should be noted that lutein is believed to be present in plant sources at much greater quantities than zeaxanthin, because the epsilon ring of lutein gives it a slightly bent configuration, at that end of the molecule. That slightly bent structure is believed to allow lutein to fit in an optimal manner into a certain type of multi-component chloroplast structure that plant cells use in photosynthesis. However, since animals do not carry out photosynthesis and do not have chloroplasts, that advantage is of no value in animal cells, and the offsetting advantages of zeaxanthin, as described above, become of greater importance.

Because of these structural, conjugational, UV-absorbing, anti-oxidant, and depositional differences between zeaxanthin and lutein, it is believed that zeaxanthin is preferable to lutein, in reducing light hyper-sensitivity. Nevertheless, because lutein is deposited in the macula, and because it is known to have substantial efficacy in absorbing UV radiation and quenching oxidative free radicals, lutein is also expected and anticipated to be able to provide at least some benefits, in reducing light hyper-sensitivity. Accordingly, lutein supplements can be orally ingested, if desired, at dosages ranging from about 10 to about 100 mg/day, either alone or in combination with zeaxanthin, to reduce and control light hyper-sensitivity.

Isomers and Esters

It should be noted that (i) various isomers of zeaxanthin exist, and (i) the isomer that is present naturally, in food sources such as corn and spinach, is presumed and believed to be preferable to other isomers.

Briefly, such isomers fall into two different categories. One category relates to “stereoisomers”, which involve the orientation of the two hydroxy groups on the end rings of zeaxanthin. Briefly, this orientation can be thought of in terms of whether the hydroxy groups would point up, or down, if the ring structure is in a flat horizontal orientation.

The only isomer of zeaxanthin that has even been found in plant sources is 3R, 3′R zeaxanthin. Accordingly, it is preferred for use as disclosed herein. Although a report appeared in 1986 (Maoka et al 1986) indicating that meso-zeaxanthin had been found in the skin of certain types of fish, subsequent research reported by Frederick Khachik and others (Khachik et al 2002) indicated that any such meso-zeaxanthin reported by Maoka et al may have resulted, instead, from the chemical extraction and analytical reagents and methods that were used by Maoka et al to extract and analyze carotenoids from those fish.

Another category of isomers, in carotenoids, relates to whether the center-chain portion, which connects the two end rings, is straight or bent. In natural dietary zeaxanthin, the chain has a consistently “trans” or “E” configuration which keeps the zig-zagging structure shown in FIG. 1 in an overall straight configuration, which gives the chain its maximum possible length. However, some types of chemical processing can give zeaxanthin a bent and crooked center-chain configuration. When this occurs, the chain is referred to as having a “cis” or “Z” configuration. These are generally undesirable, and are less preferred than trans (or E) isomers.

One of the goals of ZeaVision is to provide consumers with the best possible treatments, for eye-related conditions such as macular degeneration and light hyper-sensitivity. Therefore, 3R, 3′R stereoisomers, and all-trans, all-E center chains, are strongly preferred for any such use. However, not all companies that sell nutritional supplements adhere to those goals, and several companies sell products that are known or believed to contain meso-zeaxanthin rather than the dietary R,R-zeaxanthin, or that are believed likely to contain bent center chains having the cis (or Z) configuration, created by certain types of chemical processing. Accordingly, in order to provide ZeaVision with a legal right to prevent such companies from selling less-than-preferred isomers to unsuspecting consumers, the claims herein are deliberately intended to cover any and all stereoisomers of zeaxanthin, and any and all bent-chain “cis” isomers of zeaxanthin.

It should also be noted that some plant sources generate zeaxanthin and/or lutein in the form of esters, having one or more types of carboxylic acids bonded to the hydroxy groups on the end rings, in a common naturally occurring type of bond known as an ester bond. These bonds are commonly broken, by esterase enzymes, soon after such carotenoid esters are ingested, releasing the “free” or “alcohol” forms of zeaxanthin or lutein as illustrated in FIG. 1. Therefore, for all purposes herein, any such esters are regarded as being functionally and nutritionally equivalent to the free carotenoids.

In addition, this invention also discloses zeaxanthin and/or lutein supplements that have labels or other packaging that explicitly state that the products therein are effective in reducing light hyper-sensitivity (and/or one or more categories thereof, or medical conditions that are triggered or aggravated by light hyper-sensitivity, such as photophobia, photoreactive epileptic seizures, light-triggered migraine headaches, etc.). As legislated by Congress and as regulated by the Food and Drug Administration, any drugs that are used to treat medical conditions can be sold only when packaged with certain labeling requirements that set forth accurate information about the drug. Accordingly, under the laws administered by the FDA, the drug-plus-label combination must be sold as a single indivisible item of commerce. Accordingly, that type of tangible and indivisible item of commerce, containing zeaxanthin and/or lutein supplements packaged in a container containing a label that explicitly informs purchasers that the zeaxanthin and/or lutein supplements in the package are effective in reducing light hyper-sensitivity, if taken orally at recommended dosages (such as at least about 10 mg/day, for people suffering from adverse effects of light hyper-sensitivity) is regarded as an article of manufacture that is covered by this invention.

Thus there has been shown and described novel means for reducing and controlling light hyper-sensitivity, by oral ingestion of zeaxanthin and/or lutein at effective dosages. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A method for reducing light hyper-sensitivity, comprising the step of administering, to a person in need of such treatment, zeaxanthin at a dosage that reduces such light hyper-sensitivity, to an extent that is noticeable and apparent to such person.

2. The method of claim 1, wherein the zeaxanthin is administered by means of oral ingestion of unit formulations of zeaxanthin at a dosage of at least about 10 mg/day, while macular pigment optical density is increasing.

3. The method of claim 1, wherein the zeaxanthin is administered orally at a first dosage level that initially creates a higher level of macular pigment optical density in the person, followed by a second dosage level that sustains the higher level of macular pigment optical density.

4. The method of claim 1 wherein the zeaxanthin is administered orally by means selected from the group consisting of:
(a) capsules, tablets, or other unit dosage formulations;
(b) foods or beverages that have been fortified by additional zeaxanthin; and,
(c) food additives that are designed to be added to foods or beverages.

5. The method of claim 1 wherein the person is also administered at least one additional active agent, selected from the group consisting of lutein, extracts from bilberries, wolfberries, gingko biloba, or green tea, Vitamins C or E, zinc, and plant flavonoids, isoflavones, polyphenols, and anthocyanins.

6. A method for reducing light hyper-sensitivity in a person in need of such treatment, comprising the step of orally ingesting zeaxanthin, at a dosage that reduces such light hyper-sensitivity to an extent that is noticeable and apparent to such person.

7. The method of claim 6, wherein the zeaxanthin is administered by means of oral ingestion of unit formulations of zeaxanthin at a dosage of at least about 10 mg/day, while macular pigment optical density is increasing.

8. The method of claim 6, wherein the zeaxanthin is administered orally at a first dosage level that initially creates a higher level of macular pigment optical density in the person, followed by a second dosage level that sustains the higher level of macular pigment optical density.

9. The method of claim 6, wherein the zeaxanthin is administered orally by means selected from the group consisting of:
(a) capsules, tablets, or other unit dosage formulations;
(b) foods or beverages that have been fortified by additional zeaxanthin; and,
(c) food additives that are designed to be added to foods or beverages.

10. The method of claim 6 wherein the person is also administered at least one additional active agent, selected from the group consisting of lutein, extracts from bilberries, wolfberries, gingko biloba, or green tea, Vitamins C or E, zinc, and plant flavonoids, isoflavones, polyphenols, and anthocyanins.

11. A method for reducing adverse medical conditions relating to light hyper-sensitivity, comprising the step of orally ingesting zeaxanthin, at a dosage that significantly reduces such adverse medical conditions.

12. The method of claim 11, wherein zeaxanthin ingestion causes a significant reduction in frequency or severity of headaches.

13. The method of claim 11, wherein zeaxanthin ingestion causes a significant reduction in frequency or severity of epileptic seizures.

14. The method of claim 11, wherein zeaxanthin ingestion causes a significant reduction in frequency or severity of fatigue syndromes or episodes.

15. An article of manufacture, comprising a labeled package containing zeaxanthin in an orally ingestible formulation, wherein the package contains information and instructions indicating to purchasers or users that the zeaxanthin is capable of reducing one or more adverse aspects of light hyper-sensitivity, if orally ingested at recommended dosages.

16. The article of manufacture of claim 15, wherein the information and instructions on the package recommend a daily dosage of at least 10 mg/day, for people suffering from adverse effects of light hyper-sensitivity.
